(19) United States
(12) Patent Application Publication
(10) Pub. No.: US 2010/0022940 A1
(43) Pub. Date: Jan. 28, 2010

(54) Percutaneously Introduceable Shunt Devices and Methods

(75) Inventor: Dustin Thompson, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/179,802

(22) Filed: Jul. 25, 2008

(51) Int. Cl. A61F 2/06; A61F 2/00; A61K 35/00

(52) U.S. Cl. 604/9; 424/423; 424/93.7; 604/8; 623/1.13; 623/1.11

(57) ABSTRACT

Catheters, implantable shunt devices and methods usable to establish passageways between blood vessels and/or other anatomical structures within the body of a human or animal subject.

### FIELD OF THE INVENTION

The present invention relates generally to methods and apparatus for medical treatment and more particularly to catheters and implantable shunt devices usable to establish passageways between blood vessels and/or other anatomical structures.

### BACKGROUND

In modern medicine there are numerous situations in which it is desirable to create shunts or flow-through connections between blood vessels and/or other anatomical structures of the body. In many cases, open surgical techniques have been used to form anastomotic connections or fistulas between adjacent vessels of body structures. More recently, percutaneous catheter-based techniques and devices have been developed for creating channels or passageways (i.e., shunts) between adjacent vessels or anatomical structures.

### Percutaneous Catheter-Based Technology for Forming Channels Between Body Lumens

The prior art has also included certain tissue penetrating catheter devices, channel sizing devices and methods whereby shunts or flow-through connections may be made between body lumens (e.g., blood vessels) and/or anatomical structures. For example, U.S. Pat. Nos. 5,830,222 (Makower), 6,068,638 (Makower), 6,159,225 (Makower), 6,190,353 (Makower, et al.), 6,283,951 (Flaherty, et al.), 6,375,615 (Flaherty, et al.), 6,508,824 (Flaherty, et al.), 6,544,230 (Flaherty, et al.), 6,655,386 (Makower et al.), 6,579,311 (Makower), 6,602,241 (Makower, et al.), 6,655,386 (Makower, et al.), 6,660,024 (Flaherty, et al.), 6,685,648 (Flaherty, et al.), 6,709,444 (Makower), 6,726,677 (Flaherty, et al.) and 6,746,464 (Makower) describe a variety of tissue penetrating catheter devices, channel sizing devices, anastomotic connectors and other apparatus that may be used to form channels or passageways (i.e., shunts) between adjacent vessels or anatomical structures. The entire disclosure of each such patent is expressly incorporated herein by reference. One such tissue penetrating catheter having a laterally deployable penetrating needle and on-board ultrasound guidance is commercially available. (the Pioneer™ catheter available from Medtronic CardioVascular, Inc., Santa Rosa, Calif.).

### Vessel to Vessel Shunts for Bypassing Arterial Obstructions

The prior art has included surgical as well as percutaneous catheter-based techniques for creating shunts or connections between blood vessels. For example, percutaneous in-situ coronary venous arterialization (PICVA) and percutaneous in-situ coronary artery bypass (PICAB) are promising new transluminal catheter-based procedures that may be used for bypassing obstructions in arteries. In the PICAB procedure, catheter devices are used to create a first channel between a source artery (e.g., a segment of the obstructed artery upstream of the obstruction or another nearby artery) and a vein and a second channel between the vein and the obstructed artery at a location downstream of (i.e., distal to) the obstruction. Embolic blockers are placed in the vein to cause arterial blood that has entered the vein through the first channel to flow through the vein (in a direction opposite normal venous bloodflow) and to then pass through the second channel and into the diseased artery at a location downstream of the obstruction. In this manner, the PICAB procedure may be used to perform an in-situ bypass of the arterial obstruction in an artery. In the PICVA procedure, a single channel is formed to cause blood to flow from a source artery (e.g., a segment of the obstructed artery upstream of the obstruction or another nearby artery) into a vein that receives at least a portion of its venous flow from a capillary bed located in an ischemic or under-perfused area. An embolic blocker is placed in the vein to cause arterial blood that has entered the vein to flow through the vein (in a direction opposite normal venous bloodflow) so as to retro-perfuse the capillary bed with arterial blood. In this manner, the PICVA procedure causes the vein to become “arterialized” to effect perfusion of an ischemic or under-perfused area. Examples of tissue penetrating catheters, channel enlarging devices, embolic blockers and related methods and devices for performing PICAB and/or PICVA are described in a United States Patent Nos. U.S. Pat. Nos. 5,830,222 (Makower), 6,068,638 (Makower), 6,159,225 (Makower), 6,190,353 (Makower, et al.), 6,283,951 (Flaherty, et al.), 6,375,615 (Flaherty, et al.), 6,508,824 (Flaherty, et al.), 6,544,230 (Flaherty, et al.), 6,655,386 (Makower et al.), 6,579,311 (Makower), 6,602,241 (Makower, et al.), 6,655,386 (Makower, et al.), 6,660,024 (Flaherty, et al.), 6,561,998 (Roth et al.), 6,638,293 (Makower et al.), 6,685,648 (Flaherty, et al.), 6,709,444 (Makower), 6,726,677 (Flaherty, et al.) and 6,746,464 (Makower), the entire disclosures of which are expressly incorporated herein by reference.

### Arteriovenous Shunts for Vascular Access

In modern medicine, there are various treatments which require blood to be removed from a patient and passed through an extracorporeal blood circuit. Such treatments include, for example, hemodialysis, hemofiltration, hemodiafiltration, plasmapheresis, and extracorporeal membrane oxygenation (ECMO). Typically, the blood is removed from a blood vessel at an access site and returned to either the same blood vessel or at another location in the body. In the past, it has been common for the vascular access site to be a surgically created fistula between an artery and a vein and for blood to be removed from the fistula through an arterial needle and returned into the fistula through a venous needle. Another way to establish vascular access is by connection of a shunt device (e.g., a graft tube formed of biological or synthetic material) between an artery and an adjacent vein such that the removal and return needles may then be inserted into the graft. In some cases, at least a portion of the graft may be exteriorized so that needles may be inserted and removed without penetration through the skin while in other cases the graft may be implanted subcutaneously. For example, U.S. Pat. No. 3,998,222 (Shihata) describes a surgically implanted, totally subcutaneous arterio-venous valved shunt, wherein no elements of the shunt are exposed supracutaneously. In a described embodiment, this shunt device comprises a curved tubular shunt extending between an artery and a vein in a patient, with a pair of valves subcutaneously mounted in outlets in the shunt. The valves open and close fluid passages to the interior of the shunt upon axial movement of valve members. In use, a hollow needle is inserted through the patient's skin and mounted in an outlet opening in each valve member. The valve member is moved axially by the needle to open and close the valve and provide access to the blood flowing through the shunt. In a dialysis operation, blood is diverted to flow out of one (arterial) valve, through the dialyzer, and back through another (venous) valve of the subcutaneous shunt into the patient. Alternatively, one valve can be used for both outflow and inflow. On completion of dialysis, the valves are closed and the arterial and venous needles are withdrawn from the patient. When a single valve is used, a single needle suffices for both outflow and inflow.

U.S. Pat. No. 6,086,553 (Akbik) describes a shunt device that can be used for hemodialysis and other conditions where a vascular access may be needed. A soft main tube made of PTFE is used with two extension tubes. The ends of the main tube are anastomosed to an artery and a vein. The extension tubes connected to the main tube at one end are connected to the dialysis machine at an opposite end. The entire graft (main tube) is placed in the subcutaneous or deep tissues except for the two exposed ends of the extension tubes which remain in the external position allowing an easy, non-traumatic access to the blood flow.

### Arteriovenous Shunts for Treatment of Chronic Obstructive Pulmonary Disease (COPD)

The approach is to create an arteriovenous fistula by implanting a shunt-like device between two major leg blood vessels, utilizing cardiovascular reserve to overcome respiratory insufficiency and improve oxygenation to the lungs. The implantation of the shunt can increase cardiac output by about one liter per minute, without impacting heart rate or oxygen consumptions Instead, Dr. Sievert said the treatment increases venous oxygen content and arterial oxygen content. In the procedure, clinicians perform simultaneous arterial and venous angiograms to locate the region where the femoral artery and the iliac lie near each other in the leg. The vein is punctured and then the artery is punctured. A 5-mm-wide stent-like shunt connects the blood vessels, creating the fistula.

### Aortico-Pulmonary Shunts for Treatment of Congenital Heart Defects

Certain congenital heart defects can cause obstruction of pulmonary blood flow and right-to-left shunting of blood, resulting in cyanosis of the newborn infant (i.e., commonly known as the “blue baby” syndrome). One common congenital defect of this type is Tetralogy of Fallot which is characterized by a ventricular septal defect (a hole in the septum between the ventricles) in combination with some degree of flow obstruction between right ventricle and the lungs (i.e., pulmonary artery stenosis). Conventional methods for treating this condition involve the surgical creation of a passageway between the aorta and the pulmonary artery (e.g., an aortico-pulmonary shunt) with the objective of increasing pulmonary blood flows improved oxygenation, and relief of cyanosis. As an alternative to surgical intervention, catheter-based techniques for creating an aortico-pulmonary shunt have been devised. For example, U.S. Pat. No. 5,297,564 (Love) describes a method wherein a catheter is introduced into the body and positioned within the pulmonary artery or aorta at a location where the pulmonary artery and aorta form a common trunk. A laser is then delivered through the catheter to create an opening (i.e., a fistula) between the aorta and the pulmonary artery. The catheter may also be used to monitor hemodynamic variables and oxygenation after the laser has been used to form an initial fistula. Thereafter, the laser may optionally be employed to increase the size of the fistula until the monitored variables and oxygenation are at desired levels.

### Peritoneovenous and Peritoneourinary Shunts for Treatment of Ascites

Ascites, often contributes to morbidity and discomfort in cancer patients. In cases where medical management is inadequate, other interventions such as paracentesis, implantation of drainage ports or implantation of shunts to divert the ascitic fluid into the urinary bladder have been employed. Also, in some cases, a peritoneovenous shunt may be implanted to carry ascetic fluid from the peritoneal cavity into the venous circulation. These peritoneovenous shunts have heretofore been implanted by open surgical technique or under radiological guidance. Hussain, Fuad F.; Peritoneovenous Shunt Insertion for Intractable Ascites: A District General Hospital Experience; Cardiovasc. Intervent. Radiol.; Vol. 27, Pages 325-328 (2004).

There remains a need in the art for the development of additional devices and catheter-based methods for creating channels or passageways (i.e., shunts) between adjacent vessels or anatomical structures without the need for open surgery.

### SUMMARY OF THE INVENTION

In accordance with one aspect of the present invention, there is provided an implantable shunting device. Such device generally comprises (A) a tube portion that has a lumen, a first end and a second end and is expandable from a collapsed configuration to an expanded configuration of a first diameter; (B) a first anchoring member attached to the first end of the tube portion, such first anchoring member being expandable from a collapsed configuration to a generally bulbous expanded configuration having a diameter larger than the expanded first diameter of the tube portion and having a plurality of openings therein to allow fluid to flow therethrough; and (C) a second anchoring member attached to the second end of the tube member, such second anchoring member being expandable from a collapsed configuration to a generally bulbous expanded configuration having a diameter larger than the expanded first diameter of the tube portion and having a plurality of openings therein to allow fluid to flow therethrough.

Further in accordance with the present invention, there is provided a method for forming a connection between a first lumen or cavity of the body of a human or animal subject and a second lumen or cavity of the subject's body. This method generally comprises the steps of: (A) providing an implantable shunting device that comprises: (i) a tube member that has a lumen, a first end and a second end, said tube being expandable from a collapsed configuration to an expanded configuration of a first diameter; (ii) a first anchoring member attached to the first end of the tube member, said first anchoring member being expandable from a collapsed configuration to a generally bulbous expanded configuration having a diameter larger than said first diameter and a plurality of openings therein to allow fluid to flow therethrough; and (iii) a second anchoring member attached to the second end of the tube member, said second anchoring member being expandable from a collapsed configuration to a generally bulbous expanded configuration having a diameter larger than said first diameter and a plurality of openings therein to allow fluid to flow therethrough; (B) forming a penetration tract from the first lumen or cavity to the second lumen or cavity; (C) advancing the shunting device through the penetration tract while the tubular member, first anchoring member and second anchoring member are in their collapsed configurations, to a position where the first anchoring member is in the first lumen or cavity of the body, the second anchoring member is in the second lumen or cavity and the tube member extends through the penetration tract; and (D) causing the tubular member, first anchoring member and second anchoring member to expand to their expanded configurations.

Further aspects, details and embodiments of the present invention will be understood by those of skill in the art upon reading the following detailed description of the invention and the accompanying drawings.

### BRIEF DESCRIPTION OF THE DRAWINGS

FIG. 1 is a side view of one embodiment of a tissue penetrating catheter device useable in the system and method of the present invention.

FIG. 2 is a perspective view of one embodiment of a shunt device of the present invention.

FIG. 2A is an enlarged view of region 2A of FIG. 2.

FIG. 2B is a partial perspective view of another embodiment of a shunt device of the present invention.

FIG. 2C is a partial perspective view of yet another embodiment of a shunt device of the present invention.

FIG. 2D is a partial perspective view of the shunt device of FIG. 2 wherein the anchoring members are pressure expandable and a balloon is being used to expand one of the pressure-expandable anchoring members within the lumen of an anatomical structure such as a blood vessel.

FIG. 2E is a partial perspective view of the shunt device of FIG. 2b wherein the anchoring members are at least partially plastically deformable and a balloon catheter has been advanced into one of the anchoring members and is being used to plastically deform the anchoring member in situ within the lumen of a luminal anatomical structure such as a blood vessel.

FIGS. 3A-3H show steps in a percutaneous catheter-based method wherein the penetrating catheter of FIG. 1 is used to facilitate implantation of the shunt device of FIGS. 2-2A.

### DETAILED DESCRIPTION

The following detailed description and the accompanying drawings are intended to describe some, but not necessarily all, examples or embodiments of the invention. The contents of this detailed description and accompanying drawings do not limit the scope of the invention in any way.

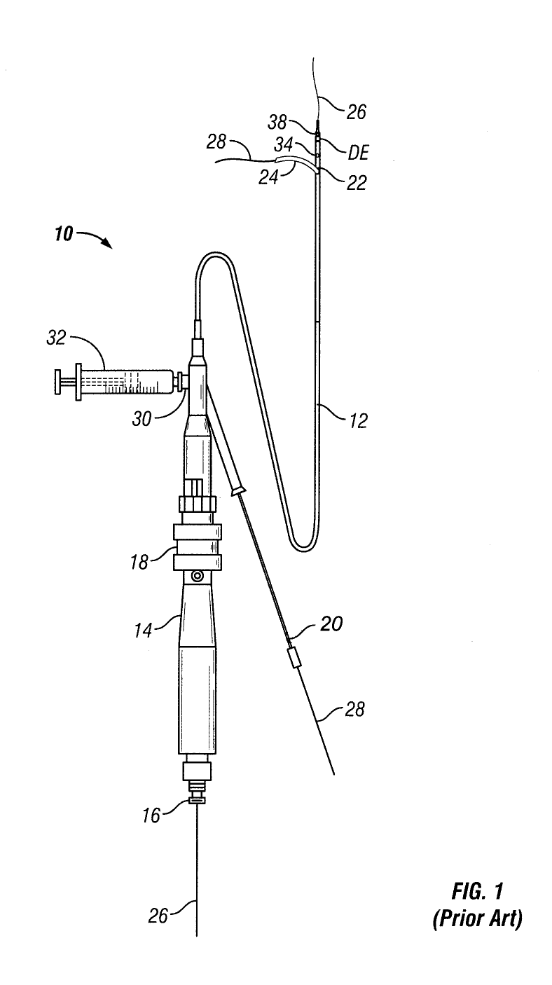

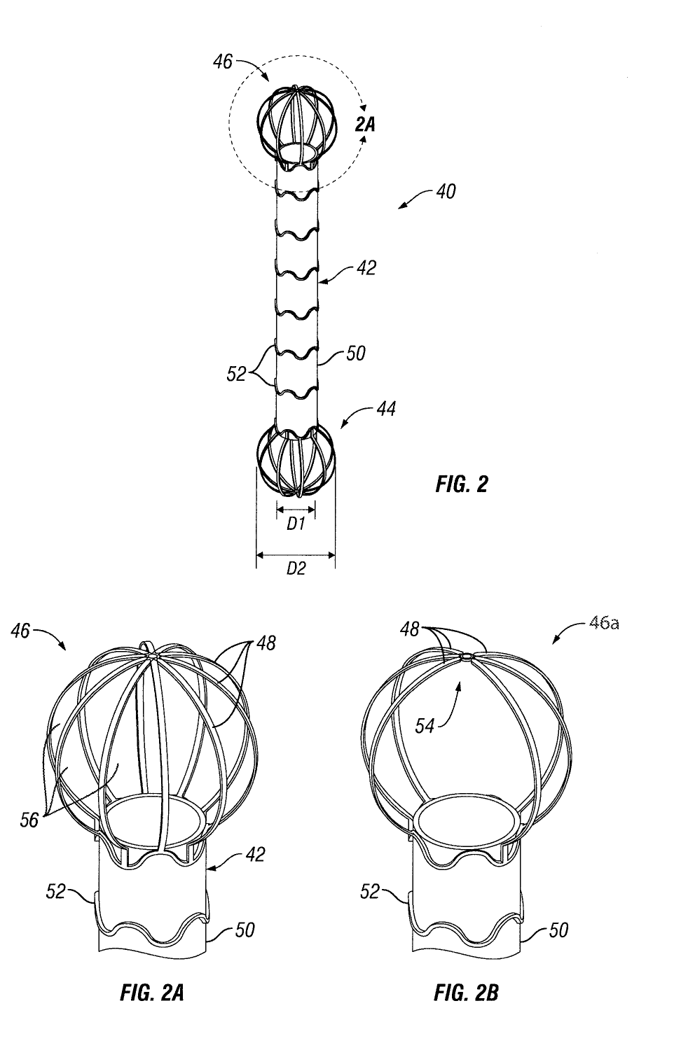

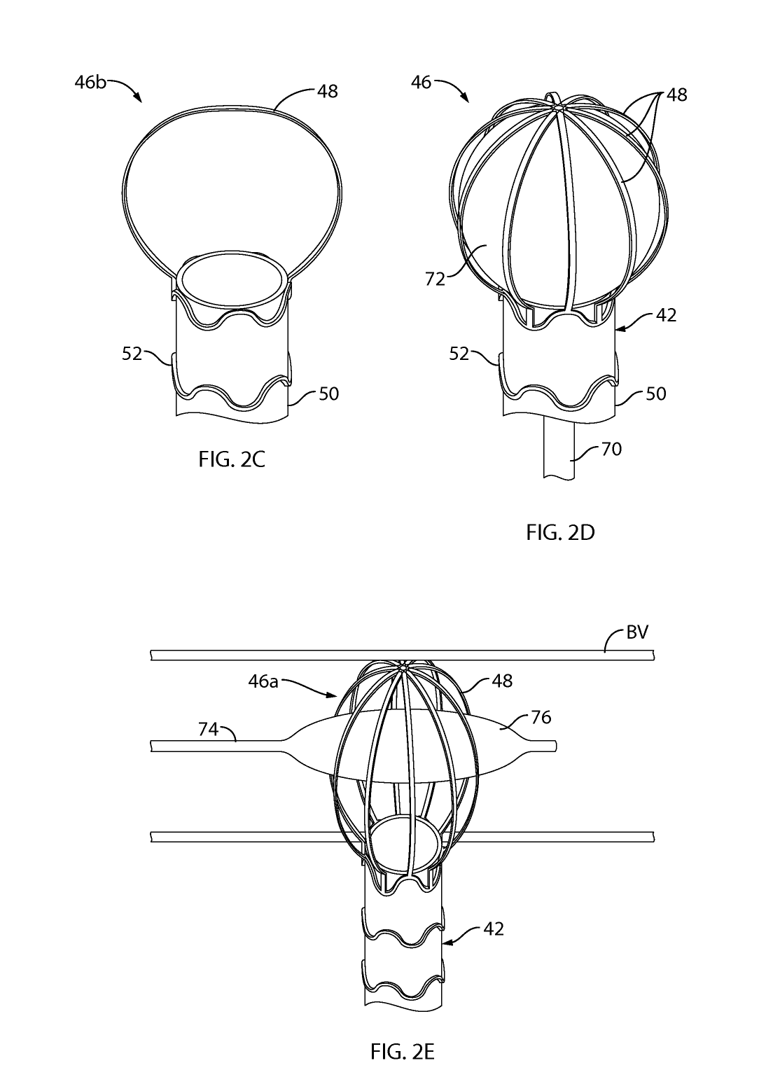

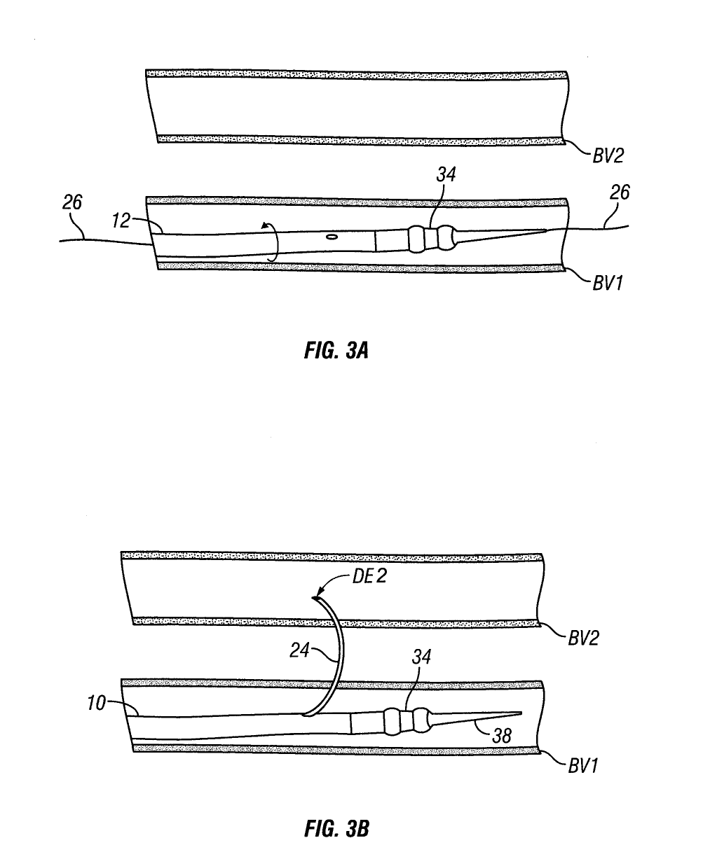

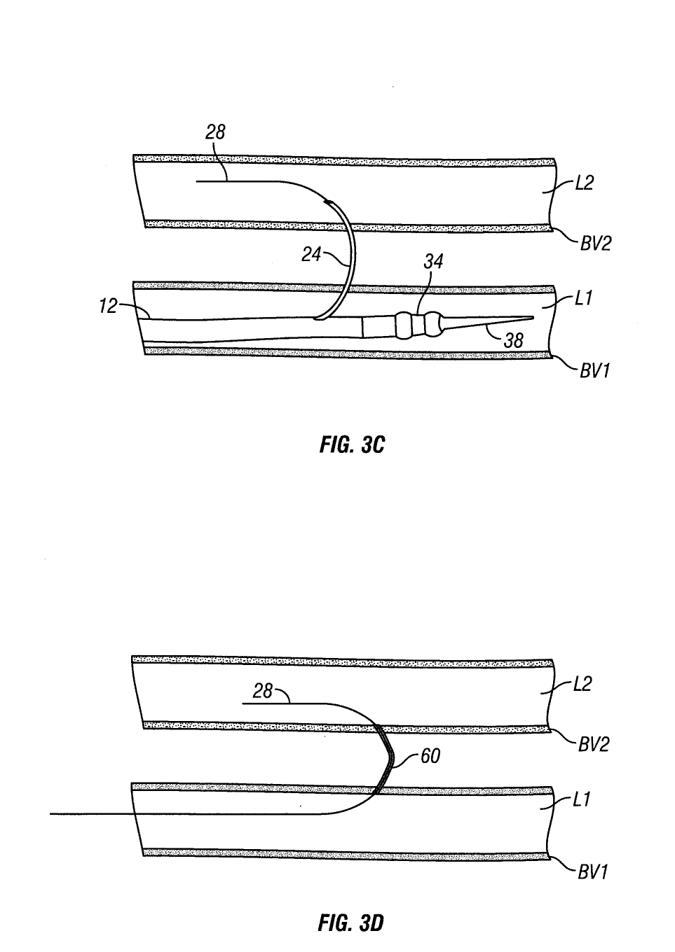

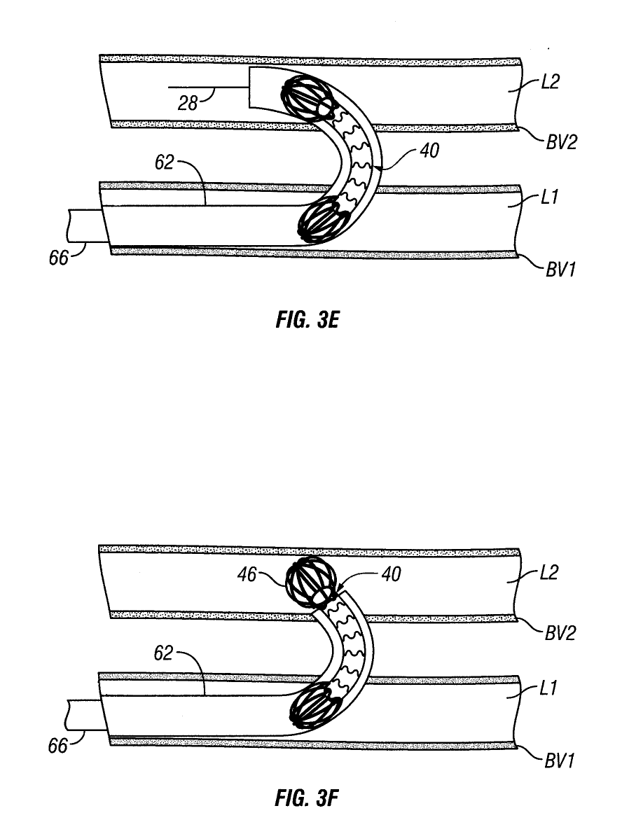

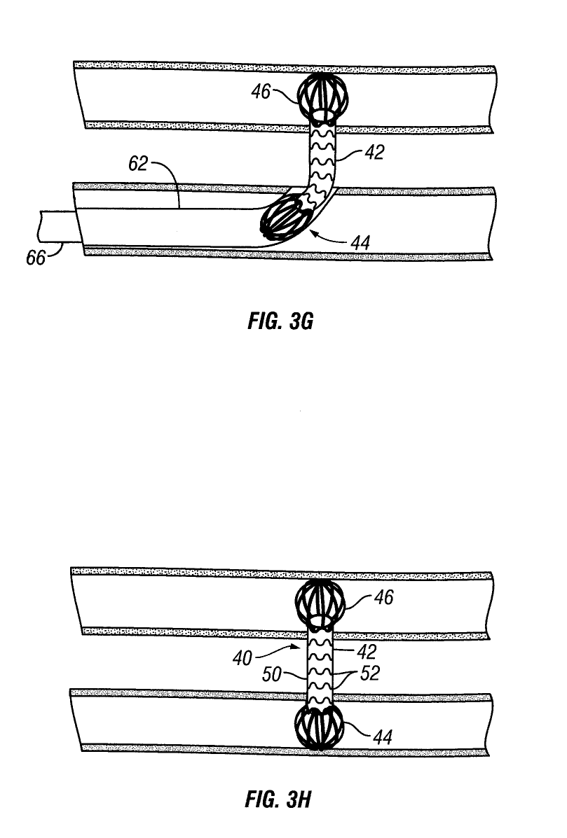

FIG. 1 shows one example of a tissue penetrating catheter device 10 that may be used to facilitate implantation of the shunt device 40 of the present invention. FIGS. 2 through 2D show examples of shunt device 40 of the present invention. FIGS. 3A-3H show one example of a method that may be used for implantation of the shunt devices of the present invention using the tissue penetrating catheter 10 seen in FIG. 1.

With reference to FIG. 1, there is shown a tissue penetrating catheter device 10 comprising an elongate catheter shaft 12 that extends distally from a handpiece 14. A side port 22 is formed in the catheter shaft 12. A penetrator advancement/retraction control 18 is useable to move a tissue penetrator 24 back and forth between a retracted position wherein the tissue penetrator 24 is within the catheter body 12 and an extended position wherein the tissue penetrator 24 extends out of the side port and in a generally away from the catheter body 12 (see FIG. 1). Optionally, an orientation apparatus 34 may also be provided on or in the catheter body 12. This orientation apparatus 34 may, in some embodiments, comprise an imageable marker (e.g., a radiopaque pointer or arrow) which may be imaged by a separate imaging device (e.g., a separate fluoroscope, x-ray, MRI, etc.) to indicate the predicted trajectory on which the tissue penetrator 24 will advance from the catheter shaft 12. In other embodiments, this orientation apparatus 34 may comprise an on-board imaging apparatus (e.g., an ultrasound transducer, optical coherence device, etc.) located on or in the catheter body 12 in combination with a physical or electronic (e.g., virtual) marker that creates, on an image received from the on-board imaging apparatus, an indication of the predicted trajectory on which the tissue penetrator 24 will advance from the catheter body 12 while the tissue penetrator 24 is still in its retracted position. By use of the orientation apparatus 34, the operator is provided with an image or indication of the intended target location along with an indication of the predicted trajectory on which the tissue penetrator 24 will subsequently advance from the catheter body 12. Initially, if the predicted penetrator trajectory is not properly aligned with the intended target location, the operator may then adjust the position and/or rotational orientation of the catheter shaft 12 within the subject's body as needed to cause the projected penetrator trajectory to become properly aligned with the intended target location. Thereafter, the operator may use the penetrator advancement/retraction control 18 to advance the penetrator 24 to the target location. Specific examples and details of tissue penetrating catheters that incorporate orientation apparatus 34 and their methods of use are described in United States Patent Nos. U.S. Pat. Nos. 5,830,222 (Makower); 6,068,638 (Makower), 6,159,225 (Makower), 6,190,353 (Makower, et al.), 6,283,951 (Flaherty, et al.), 6,375,615 (Flaherty, et al.), 6,508,824 (Flaherty, et al.), 6,544,230 (Flaherty, et al.), 6,655,386 (Makower et al.), 6,579,311 (Makower), 6,602,241 (Makower, et al.), 6,655,386 (Makower, et al.), 6,660,024 (Flaherty, et al.), 6,685,648 (Flaherty, et al.), 6,709,444 (Makower), 6,726,677 (Flaherty, et al.) and 6,746,464 (Makower) and co-pending United States Patent Applications having Ser. Nos. 11/279,993; 11/279,265; 11/279,771; 11/610,092; 11/534,895; 11/613,764; 11/837,718; 12/054,533 and 12/045,120, the entire disclosure of each such patent and patent application being expressly incorporated herein by reference. Also, there exists a commercially available tissue penetrating catheter of this type which includes an on-board ultrasound imaging transducer in combination with a marker that provides an image of the target location along with an indication of the projected penetrator trajectory relative to the target location (i.e., the Pioneer™ Catheter, Medtronic CardioVascular, Inc., Santa Rosa, Calif.).

In some embodiments of the tissue penetrating catheter 10, a guidewire lumen may be provided to allow the catheter body 12 to be advanced over a previously inserted guidewire 26. In the particular example shown, the guidewire lumen extends through the device 10 from a port 16 on the proximal end of the handpiece 14 to an outlet opening in the distal end DE of the catheter body 12. A Touhy Borst adapter or other valve (e.g., a one way valve) may be provided on or near port 16 to close the port 16 when no guidewire extends therethrough and/or to form a seal around the guidewire 26, thereby preventing fluid from escaping or backflowing out of port 16. Optionally, a second port 30 such as Luer connector may also communicate with the guidewire lumen and an infusion or aspiration device 32 such as a syringe or other suitable infusion or aspiration apparatus (e.g., a pump, solution administration tube attached to I.V. bag or bottle, suction tube, etc.) may be attached to the second port 30 and used to infuse substances (e.g., radiographic contrast medium, drugs or therapeutic substances, saline solution, oxygenated perfusate, etc.) and/or aspirate matter, when so desired. Although FIG. 1 shows an over-the-wire embodiment of the catheter wherein the guidewire 26 is received within a lumen that extends through the entire length of the catheter device, it is to be appreciated that this guidewire lumen need not necessarily extend all the way through the catheter. In alternative embodiments, a “rapid exchange” type guidewire lumen may be provided wherein a guidewire exit port is formed in the side of the catheter body and the guidewire 26 extends only through a distal portion of the catheter body. One such embodiment of the penetrating catheter is currently commercially available (Pioneer™ Catheter, Medtronic CardioVascular, Inc., Santa Rosa, Calif.).

Also, in the example of the tissue penetrating catheter 10 shown in FIG. 1, the tissue penetrator 24 comprises a hollow needle having a lumen in communication with a second guidewire port 20. This allows a second guidewire 28 to be advanced through the penetrator 24 and out of an opening in the distal end of the penetrator 24.

FIGS. 2 and 2A show one embodiment of an implantable shunt device 40 of the present invention. In this embodiment, the shunt device 40 comprises a radially expandable tubular graft portion 42 having expandable anchoring members 44, 46 on either end thereof. In this particular example, the tubular graft portion 42 comprises flexible stent graft made of a flexible tube 50 with a plurality of radially expandable support members 52 attached to the flexible tube 50 at spaced-apart locations.

In this example, the flexible tube may be formed of a natural material (e.g., fixed bovine pericardium, etc.) or a polymeric material (e.g., polytetrofluoroethylene (PTFE), expanded polytetrofluoroethylene (e-PTFE), woven polyester mesh, etc.) Also, in this example, each radially expandable support member 52 comprises a self-expanding zig-zag ring formed of elastic or superelastic material, such as a nickel-titanium alloy (Nitinol). Each support member 52 is biased to an expanded configuration of diameter D₁. As described more fully herebelow, the tubular graft portion 42 may be compressed and constrained in a radially collapsed state but, when unconstrained, the tubular graft portion 42 will assume an expanded configuration of diameter D₁ as seen in FIG. 2. In other embodiments, the support member(s) 52 may be plastically deformable such that a balloon or other expandable member may be positioned within the tube 52 while the support member(s) 52 is/are in radially collapsed or crimped configurations and a balloon or other expandable member may then be used to pressure-expand the flexible tube 50 and the support member(s) 52, causing the support member(s) 52 to plastically deform to the expanded diameter D₁. In such pressure-expandable embodiments, an opening may be formed on one end of at least one of the expandable anchoring members 44 or 46 to allow a balloon or other expandable member to be inserted into and removed from the inner lumen of the expandable graft portion 42.

It is to be appreciated that, although the drawings show an embodiment wherein separate support members 52 are at spaced-apart locations along the length of the flexible tube 50, in other embodiments the flexible tube 50 may be supported by a unitary stent structure as opposed to a series of unconnected support members 42.

In the example shown in the drawings, the expandable anchoring members 44 or 46 comprise self-expanding cages formed of generally arcuate members 48 in a circumferential arrangement such that each anchoring member 44, 46 may be initially compressed and constrained in a collapsed configuration and subsequently allowed to self-expand (when unconstrained) to an expanded configuration of diameter D₂. In such self-expanding embodiments, the arcuate members 48 may be formed of elastic or superelastic material, such as a nickel-titanium alloy (Nitinol), which is biased to the expanded configuration of diameter D₂ but which may be compressed and constrained in a collapsed configuration having a diameter smaller than diameter D₂. When fully expanded, the anchoring members 44, 46 of this example form generally bulbous cage structures, as shown. In embodiments where one or both of the anchoring members 44, 46 are intended for implantation within a body lumen through which body fluid flows (e.g., a blood vessel, bile duct, urethra, etc) such anchoring member(s) 44 and/or 46 may have openings or fenestrations through which the body fluid may flow. For example, in the embodiment shown in FIGS. 2 and 2A, body fluid may flow through the open areas 56 between the arcuate members 48. FIG. 2B shows an alternative embodiment of the device wherein the arcuate members 48 do not extend about the full circumference of the anchoring member 46a, but rather only on two sides of the member such that a substantially open flow channel 54 is provided through which body fluid may flow in substantially unobstructed fashion with minimal turbulence and minimal creation of turbulence within the flowing body fluid. FIG. 2C is a partial perspective view of yet another embodiment of a shunt device of the present invention having at least one anchoring member 46b which comprises a single generally arcuate member 48 which, when expanded, forms a ring that may be oriented within the lumen of a luminal anatomical structure, such as a blood vessel, such that the member 48 extends substantially in contact with the surrounding luminal wall thereby avoiding any substantial obstruction of natural body fluid flow through the luminal anatomical structure.

Although in these examples the anchoring members 44, 46, 46a, 46b are self-expanding, it is to be appreciated that in other embodiments, the anchoring members may be formed from non-superelastic materials (e.g. stainless steel, cobalt chromium, platinum, or a cobalt-chromium-nickel alloy (Elgiloy)) initially crimped or compressed in a collapsed configuration and subsequently plastically deformable to an expanded configuration. This may be accomplished by a positioning of a balloon or other expandable member within the interior of each collapsed anchoring member 44, 46, 46a, 46b and using such balloon or expandable member to pressure-expand the anchoring members 44, 46, 46a, 46b causing them to plastically deform to the expanded diameter D₂. In such pressure-expandable embodiments, an opening may be formed on one end of at least one of the expandable anchoring members 44 or 46, 46a, 46b to allow a balloon or other expandable member to be inserted into and removed from the interiors of the anchoring members 44 or 46, 46a, 46b. By way of example, FIG. 2D shows a partial perspective view of the shunt device of FIG. 2 wherein a balloon catheter 70 having a round balloon 72 has been advanced through the shunt device and is being used to dilate the distal anchoring member 46. The same balloon 72 may then be deflated, retracted to a position within the proximal anchoring member (not shown in the partial view of FIG. 2D) and thereafter reinflated to expand the proximal anchoring member.

Also, in some embodiments, one or both anchoring members 44, 46, 46a, 46b may be at least partially plastically deformable to allow their configuration to be modified to accommodate anatomical considerations (e.g., to minimize obstruction or introduction of turbulence in body fluid that flows through a luminal anatomical structure in which that anchoring member is positioned). For example, FIG. 2E shows the shunt device of FIG. 2B wherein at least one of the anchoring members 46a is capable of being deformed in situ. In this example, the anchoring member 46a has been positioned within the lumen of a blood vessel BV and expanded to the diameter of the blood vessel lumen. Thereafter, a balloon catheter 74 has been advanced through that blood vessel BV lumen to a position where its balloon 76 is positioned within the generally arcuate members 48 of the anchoring member 46a. The balloon 76 has then been inflated to plastically deform the generally arcuate members 48 to compress them against the surrounding blood vessel wall. This step may minimize obstruction to blood flow or turbulence creation and may, in at least some patients, minimize the potential for thrombus formation and/or the need for long term anticoagulant therapy following implantation of the shunt device.

FIGS. 3A-4G show an example of a procedure in which the above-described catheter device 10 and the implantable shunt device 40 are used to establish a flow-through shunt between a first blood vessel BV1 and a second blood vessel BV2.

As seen in FIG. 3A, a first guidewire 26 is initially advanced into the lumen of the first blood vessel BV1. A distal portion of the tissue penetrating catheter body 12 is then advanced, with its tissue penetrator 24 in the retracted position, over the first guidewire 26 to a position adjacent the location where the shunt is to be created. The optional orientation apparatus 34, if present, may be used by the operator to adjust the position and rotational orientation of the catheter body 12 within the lumen of the first blood vessel BV1, while the tissue penetrator 24 remains in its retracted position, to ensure that when the tissue penetrator 24 is subsequently advanced, it will advance on the desired trajectory toward the target location (in this example—the second blood vessel BV2) and not in some other direction.

Thereafter, as shown in FIG. 3B, the tissue penetrator 24 is advanced from its retracted position to its extended position, creating a penetration tract 60 that extends through the wall of the first blood vessel BV1, through any intervening tissue and/or hollow space between from the first blood vessel BV1 and the second blood vessel BV2, through the wall of the second blood vessel BV2 and into the lumen of the second blood vessel BV2, as shown.

Thereafter, as shown in FIG. 3C, a second guidewire 28 is advanced through the lumen of penetrator 24 and into the lumen of the second blood vessel BV2.

Subsequently, as seen in FIG. 3D, the penetrator 24 is withdrawn to its retracted position and the tissue penetrating catheter body 12 as well as the first guidewire 26 are removed, leaving the second guidewire 28 in place such that it extends into the lumen of the first blood vessel BV1, through the penetration tract 60 and into the lumen of the second blood vessel BV2, as shown. Optionally, in some applications of this method, one or more tract modifying devices (e.g., balloon catheters, atherectomy catheters, etc.) may then be advanced over the guidewire 28 and used to enlarge (e.g., dilate, debulk, bore, etc.) the penetration tract 60 and then removed, leaving the second guidewire 28 in place. Examples of tract modifying devices and procedures of this sort are provided in U.S. Pat. Nos. 5,830,222 (Makower) and 6,561,998 (Roth et al.), the entire disclosures of which are expressly incorporated herein by reference.

Thereafter, as seen in FIG. 3E, a shunt delivery catheter 62 is advanced over the second guidewire 28. The shunt device 40 is positioned within the lumen of the deliver catheter 62, near its open distal end, with the tubular graft portion 42 and anchoring members 44, 46 in their collapsed configurations. The delivery catheter 62 is advanced to a position were its open distal end is within the lumen of the second blood vessel BV2. A push member 66 is positioned within the lumen of the delivery catheter 62, proximal to the shunt device 40 and the second guidewire 28 is removed.

As shown in FIG. 3F, the push member 66 is then maintained in a substantially fixed position as the delivery catheter 62 is retracted, thereby initially exposing the second anchoring member 46 and allowing the second anchoring member 46 to self-expand to its expanded configuration within the lumen of the second blood vessel BV2.

Thereafter, as seen in FIG. 3G, the delivery catheter is further retracted, uncovering the tubular graft portion 42 and allowing it to self-expand to its expanded configuration within the penetration tract 60 and then uncovering the first anchoring member 44 and allowing it to self-expand within the lumen of the first blood vessel BV1.

Finally, as seen in FIG. 3H, the delivery catheter 62 and push member 66 are removed, leaving the shunt device 40 implanted within the subject's body, creating a blood flow passageway between the first blood vessel BV1 and the second blood vessel BV2.

In accordance with techniques known in the field of vascular surgery, endothelial cells of a desired type or substance(s) that promote the growth or adhesion of endothelial cells, may be disposed on the luminal surface of, or within the wall of, the tubular member 50 prior to implantation of the shunt device 40 to enhance the potential for post-implantation endothelialization and improved patency of the tubular graft portion 42. Thus may be particularly advantageous in embodiments where the tubular member 50 is formed of synthetic material such as polytetrofluoroethylene (PTFE), expanded polytetrofluoroethylene (e-PTFE), or woven polyester mesh. Examples of substances, cell types and techniques useable for endothelial seeding, endothelial sodding and/or promotion of in situ endothelialization of vascular grafts are described in U.S. Pat. Nos. 5,723,324 (Bowlin et al.); 5,714,359 (Bowlin, et al.); 5,492,826 (Townsend, et al.); 7,037,332 (Kutryk, et al.); 7,090,834 (Cunningham et al.) and United States Patent Application Publication No. 2008/0057097 (Benco, et al.), the entire disclosure of each such patent and published patent application being hereby expressly incorporated herein by reference.

It is to be further appreciated that the invention has been described hereabove with reference to certain examples or embodiments of the invention but that various additions, deletions, alterations and modifications may be made to those examples and embodiments without departing from the intended spirit and scope of the invention. For example, any element or attribute of one embodiment or example may be incorporated into or used with another embodiment or example, unless to do so would render the embodiment or example unsuitable for its intended use. Also, where the steps of a method or process are described, listed or claimed in a particular order, such steps may be performed in any other order unless to do so would render the embodiment or example not novel, obvious to a person of ordinary skill in the relevant art or unsuitable for its intended use. All reasonable additions, deletions, modifications and alterations are to be considered equivalents of the described examples and embodiments and are to be included within the scope of the following claims.

What is claimed is:

1. An implantable shunting device comprising:
a tube member that has a lumen, a first end and a second end, said tube being expandable from a collapsed configuration to an expanded configuration of a first diameter;
a first anchoring member attached to the first end of the tube member, said first anchoring member being expandable from a collapsed configuration to a generally bulbous expanded configuration having a diameter larger than said first diameter and a plurality of openings therein to allow fluid to flow therethrough; and
a second anchoring member attached to the second end of the tube member, said second anchoring member being expandable from a collapsed configuration to a generally bulbous expanded configuration having a diameter larger than said first diameter and a plurality of openings therein to allow fluid to flow therethrough.

2. A device according to claim 1 wherein the tube member comprises a flexible tube having at least one radially expandable support member attached thereto.

3. A device according to claim 2 wherein said at least one radially expandable support member comprises a stent.

4. A device according to claim 3 wherein the tube member comprises a stent graft.

5. A device according to claim 2 wherein said at least one radially expandable support member comprises a plurality of radially expandable ring members attached to the flexible tube at spaced-apart locations.

6. A device according to claim 5 wherein the radially expandable ring members are zig-zag rings.

7. A device according to claim 2 wherein the flexible tube comprises a tube formed of flexible polymer.

8. A device according to claim 2 wherein the flexible tube is formed substantially of a material selected from polytetrafluoroethylene, woven polytetrafluoroethylene, expanded polytetrafluoroethylene, woven expanded polytetrafluoroethylene, poly ester; woven polyester; polyethylene terephthalate and woven polyethylene terephthalate.

9. A device according to claim 2 wherein the flexible tube is formed substantially of a biologic material.

10. A device according to claim 2 wherein said at least one radially expandable support member is plastically deformable from its collapsed configuration to its expanded configuration.

11. A device according to claim 2 wherein said at least one radially expandable support member self-expands from is collapsed configuration to its expanded configuration.

12. A device according to claim 1 wherein cells or an endothelilization promoting substance is disposed on an inner wall of the lumen of the tubular member.

13. A device according to claim 1 wherein the first anchoring member comprises a plurality of generally arcuate members attached to the first end of the tube member and spaced-apart such that said plurality of openings comprises spaces between adjacent ones of the generally arcuate members.

14. A device according to claim 1 wherein the second anchoring member comprises a plurality of generally arcuate members attached to the second end of the tube member and spaced-apart such that said plurality of openings comprises spaces between adjacent ones of the generally arcuate members.

15. A system comprising a device according to claim 1, further in combination with a delivery catheter useable to carry the device into the body of a human or animal subject while the tubular member, first anchoring member and second anchoring member are in their collapsed configurations and subsequently useable to deploy the device within the subject's body such that the tubular member, first anchoring member and second anchoring member expand to their expanded configurations and the delivery catheter is thereafter removable leaving the device implanted within the subject's body.

16. A system according to claim 15 further in combination with:
a tissue penetrating catheter device having a catheter body that is insertable into an anatomical lumen of the subject's body a tissue penetrator having a penetrator lumen, said tissue penetrator being advanceable from the catheter body to form a penetration tract that extends from the anatomical lumen to a target location within the subject's body; and
a guidewire that is advanceable through the penetrator lumen such that the tissue penetrator may thereafter be retracted and the penetration catheter removed, leaving the guidewire in place such that the guidewire extends from the anatomical lumen to the target location.

17. A system according to claim 16 wherein the delivery catheter has a guidewire lumen and is advanceable over the guidewire.

18. A system according to claim 16 wherein the tissue penetrating catheter further comprises an orientation apparatus which provides information to enable the user to rotationally orient the catheter to the extent needed, prior to advancement of the penetrator, to ensure that the penetrator is aimed at the target location.

19. A method for forming a connection between a first lumen or cavity of the body of a human or animal subject and a second lumen or cavity of the subject's body, said method comprising the steps of:
(A) providing an implantable shunting device that comprises:
a tube member that has a lumen, a first end and a second end, said tube being expandable from a collapsed configuration to an expanded configuration of a first diameter;
a first anchoring member attached to the first end of the tube member, said first anchoring member being expandable from a collapsed configuration to a generally bulbous expanded configuration having a diameter larger than said first diameter and a plurality of openings therein to allow fluid to flow therethrough; and
a second anchoring member attached to the second end of the tube member, said second anchoring member being expandable from a collapsed configuration to a generally bulbous expanded configuration having a diameter larger than said first diameter and a plurality of openings therein to allow fluid to flow therethrough;
(B) forming a penetration tract from the first lumen or cavity to the second lumen or cavity;
(C) advancing the shunting device through the penetration tract while the tubular member, first anchoring member and second anchoring member are in their collapsed configurations, to a position where the first anchoring member is in the first lumen or cavity of the body, the second anchoring member is in the second lumen or cavity and the tube member extends through the penetration tract; and
(D) causing the tubular member, first anchoring member and second anchoring member to expand to their expanded configurations.

20. A method according to claim 19 further comprising the step of enlarging the penetration tract prior to or during performance of Step C.

21. A method according to claim 19 wherein Step B comprises:
inserting a tissue penetrating catheter into the first lumen or cavity;
advancing a penetrator from the tissue penetrating catheter and into the second lumen or cavity to form the penetration tract; and, thereafter,
withdrawing the penetrator and removing the tissue penetrating catheter.

22. A method according to claim 21 wherein the tissue penetrating catheter has an orientation apparatus to enable the user to rotationally orient the catheter, prior to advancement of the penetrator, to ensure that the penetrator is aimed at the second lumen or cavity and wherein the method further comprises:
using the orientation apparatus to rotationally orient the catheter, prior to advancement of the penetrator, to ensure that the penetrator is aimed at the second lumen or cavity.

23. A method according to claim 19 wherein the shunting device is initially disposed within, or on, a delivery catheter with the tubular member, first anchoring member and second anchoring member are in their collapsed configurations, and wherein Step C comprises:
advancing the delivery catheter through the penetration tract to a position where the first anchoring member is in the first lumen or cavity of the body, the second anchoring member is in the second lumen or cavity and the tube member extends through the penetration tract;
deploying the shunting device from the delivery catheter such that the tubular member, first anchoring member and second anchoring member expand to their expanded configurations; and
removing the delivery catheter.

24. A method according to claim 19 wherein Step B comprises:
inserting a tissue penetrating catheter into the first lumen or cavity;
advancing a penetrator that has a penetrator lumen from the penetrating catheter and into the second lumen or cavity to form the penetration tract;
advancing a guidewire through the penetrator lumen; and, thereafter
withdrawing the penetrator and removing the tissue penetrating catheter.

25. A method according to claim 24 wherein the shunting device is initially disposed within or on a delivery catheter having a guidewire lumen with the tubular member, first anchoring member and second anchoring member in their collapsed configurations, and wherein Step C comprises:
advancing the delivery catheter over the guidewire and through the penetration tract to a position where the first anchoring member is in the first lumen or cavity of the body, the second anchoring member is in the second lumen or cavity and the tube member extends through the penetration tract;
deploying the shunting device from the delivery catheter such that the tubular member, first anchoring member and second anchoring member expand to their expanded configurations; and
removing the delivery catheter and the guidewire.

26. A method according to claim 19 wherein the first lumen or cavity comprises the lumen of a blood vessel.

27. A method according to claim 19 wherein the second lumen or cavity also comprises the lumen of a blood vessel.

28. A method according to claim 19 wherein the first lumen or cavity comprises the lumen of an artery and the second lumen or cavity comprises the lumen of another artery.

29. A method according to claim 19 wherein:
one of said first and second lumens or cavities comprises the lumen of an artery; and
the other of said first and second lumens or cavities comprises the lumen of a vein.

30. A method according to claim 29 further comprising the step of blocking the coronary vein at a location which causes blood that has flowed from the artery, through the shunt device and into the lumen of the vein to subsequently flow through the vein in a direction opposite normal venous bloodflow.

31. A method according to claim 30 further comprising the steps of:
creating a second penetration tract between the vein and the lumen of an obstructed artery at a location downstream of the obstruction; and
causing blood that has flowed from the artery, through the shunt device and into the lumen of the vein to subsequently flow through the second penetration tact and into the lumen of the obstructed artery at a location downstream of the obstruction.

32. A method according to claim 19 wherein the subject suffers from cyanosis due to a congenital cardiac deformity and wherein:
one of said first and second lumens or cavities comprises the aorta; and
the other of the first and second lumens or cavities comprises pulmonary artery; and
the performance of the method creates an aorticopulmonary shunt.

33. A method according to claim 19 wherein the first lumen or cavity comprises the lumen of a coronary blood vessel and the second lumen or cavity comprises the lumen of another coronary blood vessel.

34. A method according to claim 19 wherein the first lumen or cavity comprises the lumen of a blood vessel in a lower extremity and the second lumen or cavity comprises the lumen of a neighboring blood vessel.

35. A method according to claim 19 wherein the first lumen or cavity comprises the lumen of a blood vessel in an upper extremity and the second lumen or cavity comprises the lumen of a neighboring blood vessel.

36. A method according to claim 19 wherein the tube member has a one way valve and wherein one of said first and second body lumens or cavities comprises a blood vessel lumen and the other comprises the peritoneal cavity and wherein the shunting device is placed such that the one way valve allows fluid to flow from the peritoneal cavity into the blood vessel lumen but prevents blood from flowing from the blood vessel lumen into the peritoneal cavity.

37. A method according to claim 19 wherein:
one of said first and second lumens or cavities comprises an artery; and
the other of the first and second lumens or cavities comprises a vein; and
at least a portion of the shunt device is located at an exteriorized or subcutaneous location whereby a needle may be inserted into the shunt device for vascular access.

38. A method according to claim 19 wherein the shunt device is positioned to create an arterio-venous shunt for the purpose of treating pulmonary disease.

39. A method according to claim 19 wherein the shunt device is positioned to create an aortico-pulmonary shunt to treat a congenital heart defect.
